(19) United States
(12) Patent Application Publication
(10) Pub. No.: US 2019/0247546 A1
(43) Pub. Date: Aug. 15, 2019

(54) METHOD FOR FORMING A FUNCTIONAL NETWORK OF HUMAN NEURONAL AND GLIAL CELLS

(71) Applicants: LEIBNIZ-INSTITUT FÜR POLYMERFORSCHUNG DRESDEN E.V., 01069 Dresden (DE); DEUTSCHES ZENTRUM FÜR NEURODEGENERATIVE ERKRANKUNGEN E.V., 53175 Bonn (DE)

(72) Inventors: CHRISTOS PAPADIMITRIOU, DRESDEN (DE); CAGHAN KIZIL, DRESDEN (DE); UWE FREUDENBERG, DRESDEN (DE); CARSTEN WERNER, DRESDEN (DE)

(73) Assignees: LEIBNIZ-INSTITUT FÜR POLYMERFORSCHUNG DRESDEN E.V., 01069 Dresden (DE); DEUTSCHES ZENTRUM FÜR NEURODEGENERATIVE ERKRANKUNGEN E.V., 53175 Bonn (DE)

(21) Appl. No.: 16/301,933

(22) PCT Filed: May 12, 2017
(86) PCT No.: PCT/DE2017/100408

§ 371 Date: Nov. 15, 2018

(30) Foreign Application Priority Data
May 17, 2016 (DE) 10 2016 109 068.9

(51) Int. Cl. A61L 27/52; C12N 5/0793; C12N 5/079; C12N 5/00; C12N 5/074

(52) U.S. Cl. CPC A61L 27/52; C12N 5/0619; C12N 5/0622; C12N 2501/91; C12N 5/0696; C12N 2513/00; C12N 5/0018

(57) ABSTRACT

The invention relates to a method for forming a functional network of human neuronal and glial cells, wherein the cells are introduced into a synthetic hydrogel system with the components polyethylene glycol (PEG) and heparin and are cultivated therein. The cells are introduced into the PEG heparin hydrogel system together with one of the gel components, either PEG or heparin, with which the cells were previously mixed such that the cells are already located in the hydrogel system during the formation of the three-dimensional hydrogel.

The invention relates to a method for forming a functional network of human neuronal and glial cells, also referred to hereinafter as neuronal network. The method can be used for the monitoring of the formation of the neuronal network and, in this connection, especially for the modeling of diseases which have an effect on the formation of neurons and/or neuronal networks in the human brain.

A current study by D. Y. Kim et. al. A three-dimensional human neural cell culture model of Alzheimer's disease, Nature 2014, 515, 274-278, describes genetically modified human cells which were embedded in Matrigel® from BD Biosciences in order to provide a three-dimensional thin-layer culture having an overall axial plane of not more than 0.3 mm. The differentiated neurons were viable and functional for 4 to 12 weeks. A similar study by Dawai Zhang et al., A 3D Alzheimer's disease culture model and the induction of P21-activated kinase mediated sensing in iPSC derived neurons, Biomaterials 2014 February; 35 (5), 1420-1428, reported the use of the neuroepithelial stem-cell line (It-NES) line AF22, derived from human induced pluripotent stem cells (iPSCs), and their differentiation into neuronal lines. Furthermore, a hydrogel which was prepared from PuraMatrix® from ED Biosciences and modified with 10 μg/mL laminin was used. This culture system is based on a hydrogel system crosslinked by noncovalent interactions by means of self-assembling arginine-alanine-aspartic acid-alanine (RADA single letter code) peptides. The mechanical properties of the hydrogel are adjustable only to a limited extent owing to the relatively weak noncovalent interactions and there are also no cleavage sites which are sensitive for specific enzymes. Owing to the nonseparability of cells in the BD PuraMatrix®, this culture system does not offer a cell-responsive microenvironment. A necessary hydrogel reconstruction for the growth of embedded cells can only be achieved by the nonspecific degradation of the peptides and thus by degradation of the entire hydrogel matrix. Accordingly, according in the study by Zhang et al., a 3D culturing of the cells can only be carried out over very short periods, for example 2-4 days. In the publication by Zhang, it is explicitly pointed out that “long-term culturing is technically difficult due to the low stiffness of the material”. In addition, as a result of the admixture of laminin obtained from animal sources, a poorly defined protein which is subject to the risk of impurities or batch-dependent variations in product quality becomes a component of the assay that can give rise to severe doubts about the reproducibility of the preparation (method). Furthermore, local demixing effects and inhomogeneities of the embedded cells may occur as a result of the long gelling time (gel-formation time) of the material (20-30 minutes).

S. Koutsopoulos et. al., Long-term three-dimensional neural tissue cultures in functionalized self-assembling peptide hydrogels, Matrigel and Collagen I, Acta Biomater. 2013, February; 9 (2); 5162-5169, discloses the preparation of a non-cell-responsive, self-organizing peptide similar to PuraMatrix®, wherein neuronal mouse cells were cultured. The study by J. Y. Sang, Simple and Novel Three Dimensional Neuronal Cell Culture Using a Micro Mesh Scaffold, Exp Neurobiol. 2011 June; 20 (2); 110-115, describes a three-dimensional neuronal culture with use of synthesized nylon fibers as scaffold. Neuronal cells are mixed with agarose and then plated above onto the nylon fabric. Such a technology offers an artificial environment in which cells are encapsulated in a cell-responsive environment which is not suitable for reproducing the in vivo environment of a developing brain.

U.S. Pat. Nos. 6,306,922 A and 6,602,975 A describe a photopolymerized hydrogel which is biodegradable. However, polymerizations at wavelengths close to the UV spectrum can cause cell death and DNA mutations in cell culture systems owing to the formation of free radicals. Cell damage caused by UV waves is not to be expected in the system according to the invention, since the polymerization is carried out under normal laboratory conditions and in the absence of UV light. Furthermore, because a UV-induced photopolymerization is dispensed with, it is substantially easier to use the presently described hydrogel system outside highly specialized laboratories, since there is no need for special equipment to bring about the polymerization.

It is an object of the invention to develop a modular in vitro system or method with cells of human origin, which system or method is substantially improved in comparison with the abovementioned prior publications and which system or method forms functional neuronal networks in a three-dimensional matrix. The most important assessment criterion for such a system is the quality of the neuronal network, which is intended to reproduce the in vivo situation in neuronal tissue of the central nervous system as far as possible. Moreover, the system is intended to be easy-to-handle and to offer a more secure prospect for uses without highly specialized laboratories, for example even in transplant procedures.

The object is achieved in the form of a method for forming a functional network of human neuronal and glial cells as claimed in claim 1. Further developments are specified in the dependent claims. Further claims relate to uses of the method.

According to the invention, in the method for forming a functional network of human neuronal and glial cells, the cells are introduced into a synthetic hydrogel system containing the components polyethylene glycol (PEG) and heparin and are cultured therein. In this connection, the cells are introduced into the PEG-heparin hydrogel system together with one of the gel components, either PEG or heparin, with which the cells have been previously mixed, with the result that the cells are already present in the hydrogel system during the polymerization of the three-dimensional hydrogel.

Advantageously, in the method, the human neuronal cells are cocultured with glial cells, wherein the human neuronal cells are human neuronal stem and progenitor cells or originate from a human immortalized neuronal progenitor cell line or are primary human neuronal progenitor cells obtained from the midbrain.

According to an advantageous embodiment, the human neuronal cells are human neuronal stem and progenitor cells from induced pluripotent stem cells (iPSCs) or are derived from primary human cortical cells.

The functionality of the network formed is, in particular, describable in terms of the expression of mature neuronal cortical markers, the responsiveness to neurotransmitters, for example in the form of calcium influx, and in terms of electrophysiological activity.

According to a preferred embodiment of the invention, the PEG-heparin hydrogel system is a multiple-arm polyethylene glycol (star-PEG)-containing star-PEG-heparin hydrogel system which is crosslinked via enzymatically cleavable peptide sequences, preferably peptide sequences cleavable by means of matrix metalloproteinases (MMP peptides), the result being that the star-PEG-heparin hydrogel system is cleavable and locally reconstructible. The four-arm polyethylene glycol (four-arm star-PEG) is particularly preferred as multiple-arm polyethylene glycol.

In one embodiment, the hydrogel matrix of the hydrogel is formed by a covalent crosslinking of a thiol-terminated star-PEG-peptide conjugate and of a heparin functionalized by maleimide, preferably by 4-6 maleimide groups. In this connection, the hydrogel matrix is crosslinked via a Michael addition.

Alternatively, the hydrogel matrix of the star-PEG-heparin hydrogel system is formed noncovalently from heparin and a covalent star-PEG-peptide conjugate by self-organization. In this connection, the star-PEG-peptide conjugate comprises conjugates of two or more peptides which are coupled to a polymer chain. The peptide sequence contains a repeating dipeptide motif (BA)ₙ, where B is an amino acid having a positively charged side chain, A is alanine and n is a number from 5 to 20, preferably 5 or 7.

According to a particularly advantageous embodiment of the invention, the hydrogel has variable mechanical properties, characterized by the storage modulus. Preferably, the storage modulus is variable within a range of 300-600 pascals. The range of the storage modulus can, for example, be varied by adjusting the mixing ratio of the two material components, i.e., by varying the degree of crosslinking (synonymous with varying the molar ratio of PEG to heparin), or by varying the solids content of the material components, i.e., the concentration of the polymeric starting materials, and preferably be determined by means of oscillatory rheometry.

In a preferred embodiment of the invention, the PEG-heparin hydrogel system is modified with signaling molecules and/or with functional peptide units derived from proteins of the extracellular matrix (ECM), preferably adhesion peptides.

According to a further embodiment of the invention, the human neuronal and glial cells are cocultured together with human mesenchymal stromal cells and endothelial cells, which are colocalized with the human neuronal and glial cells.

In the method, it is advantageously possible to use human neuronal stem cells which have not been genetically modified. The cells mature naturally in the hydrogel, preferably a star-PEG-heparin hydrogel, to form completely differentiated neuronal subtypes which are positive for neuronal marker proteins such as CTIP2+, SATB2+ and TAU+. Furthermore, the cultures can survive for more than 10 weeks when using a PEG-heparin hydrogel.

In the method according to the invention, the rapid hydrogel formation, i.e., 30 seconds to a maximum of 2 minutes, avoids disadvantageous local demixing effects and inhomogeneities. The rapid, robust hydrogel formation is a major advantage of the method according to the invention.

A further aspect of the invention concerns a human, neuronal, three-dimensional functional network which is obtainable in the method according to the invention. As a result of using the method, individual neurons could be linked in a three-dimensional network and exhibited physiologically relevant cellular functions. For example, in a volume of 0.18 mm³, what were developed were more than 200 subnetworks, which were in turn linked to one another. In this connection, each, subnetwork had a total number of more than 12 000 branches. In the three-dimensional cell culture system according to the invention, it was possible to detect strong network formation and neuronal branching with differentiated neurons and colocalized glial cells of differing type, as will be described later in detail in the exemplary embodiments.

The method according to the invention using the hydrogel system allows
the reproduction of the human three-dimensional network structure and of the functionality of the human neuronal network, especially with respect to morphology, cell type and differentiation,
stability of the cell cultures to be ensured, with the result that the cell cultures survive over a longer culture time and remain stable especially during the long-term analyses,
the precise adjustment or tailoring of the hydrogel matrix, the biomolecular composition and the physical properties, such as the storage modulus, with the result that various exogenous cell-instructive signals and signaling substances, for example soluble factors, components of the extracellular matrix and mechanical properties, can be tested for neuronal development and for the modeling of diseases,
a response of the formed neuronal network to active ingredients similar to under in vivo conditions and thus the provision of a possibility to replace cost-intensive animal-experiment models and, additionally, to also provide a more reliable assay for method therapeutic active ingredients,
the determination of the electrophysiological activity and membrane-channel activity of the neuronal cells,
the analysis of individual cells, for example using high-resolution images and recording techniques, in order to be able to investigate the signaling lines and neuronal circuits in a three-dimensional neuronal network,
the real-time analysis especially of development processes of the neuronal network,
array techniques, such as printing for example, and zonal heterogeneity in order to develop organ mimetics,
usability for personalized medicine using cells originating from the patient,
coculturing with endothelial cells in order to investigate the interaction of neuronal network formation and of vessel formation under in vivo-like conditions,
the removal of individual cells, which is necessary for single-cell assays, in vitro cell expansion and for transplant purposes.
transplantation of a nontoxic and biodegradable material;
long-term culturing of more than ten weeks;
quantification of changes and of the progress of neuronal networks and of the cell count.

A further aspect of the invention concerns the use of the method according to the invention for the monitoring of the formation of the neuronal network. Thus, real-time monitoring of the formation of the neuronal network is possible especially because of the transparency of the cell culture in the three-dimensional hydrogel system.

The monitoring of network formation using the method according to the invention allows quantitative analysis of the cell growth, of the length, of the number and density of branches and/or of the connectivity and/or of the electrophysiological activity of the neuronal cells within the neuronal network.

This means that the modeling of diseases which have an effect on the formation of neurons and neuronal networks in the human brain, of developmental disorders of the nervous system or of neurodegenerative diseases is also possible.

In this connection, a particularly advantageous use of the method consists in the modeling of the neurotoxicity and/or change in neuronal stem-cell plasticity that is caused by disease-relevant protein aggregates, for example amyloid β 42.

The monitoring of network formation using the method according to the invention is also suitable for testing molecules and/or active ingredients or medicaments which influence neuronal activity and/or network formation.

Further details, features and advantages of embodiments of the invention are revealed by the following description of exemplary embodiments with reference to the associated drawings, where:

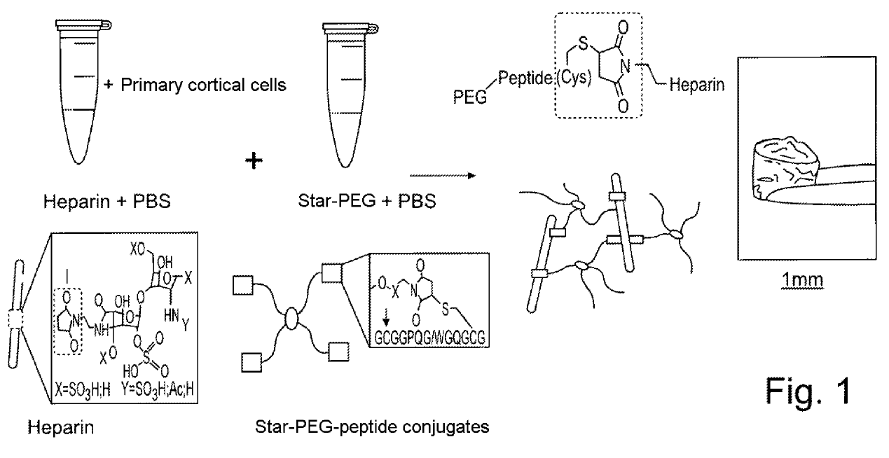

FIG. 1: shows a graphic depiction of one exemplary embodiment for the preparation of a hydrogel,

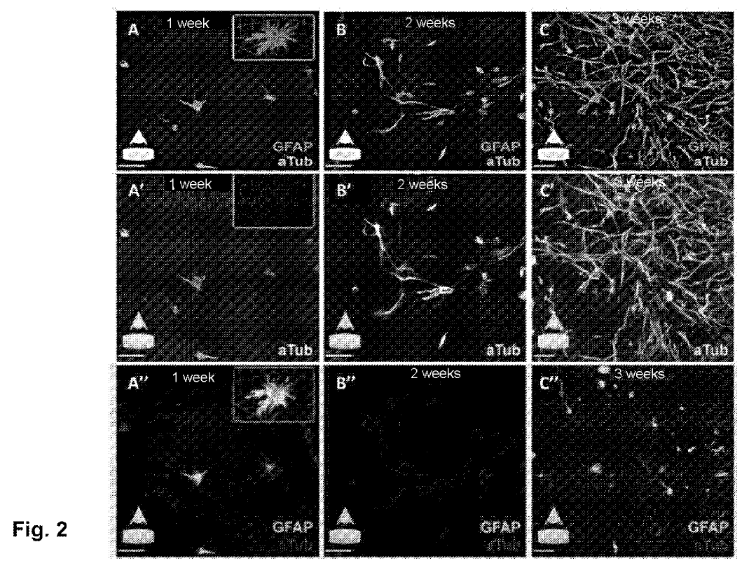

FIG. 2: shows micrographs of the maturation of the neuronal network over a period of three weeks,

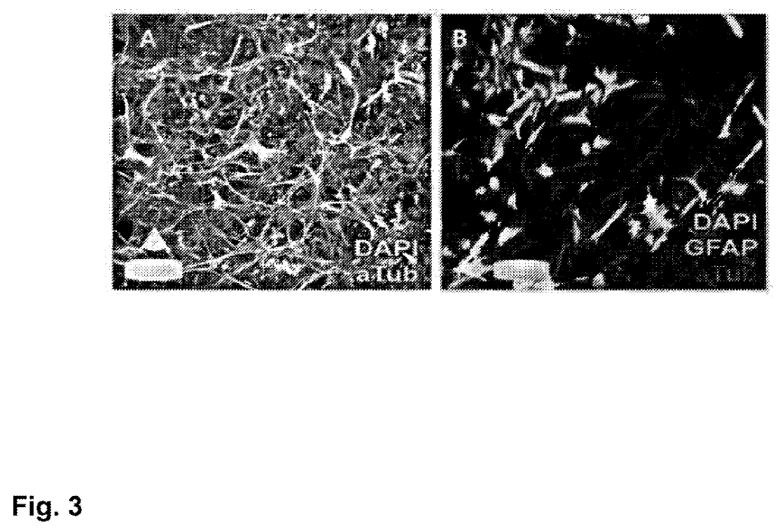

FIG. 3: shows an extensive three-dimensional depiction of a three-week-old gel containing neuronal and glial networks of high density,

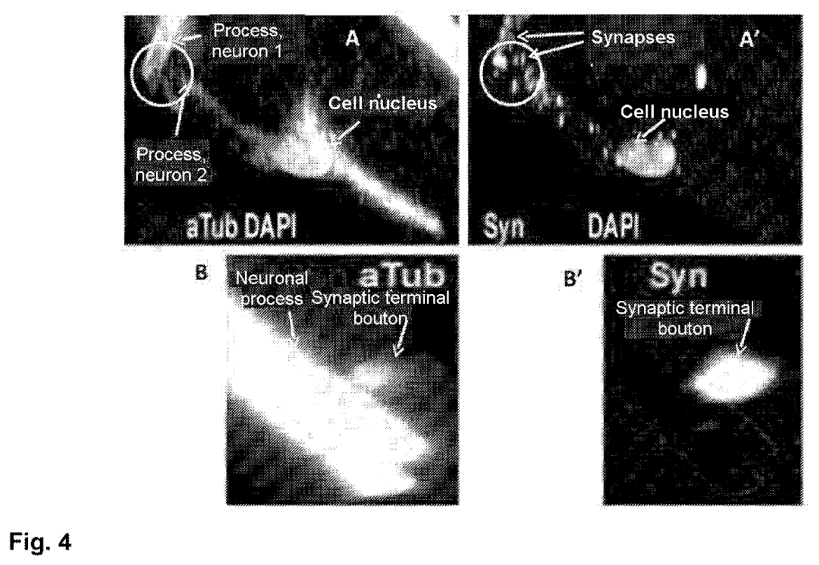

FIG. 4: shows the immunoreactivity of encapsulated neurons with respect to synaptophysin (Syn) and acetylated tubulin (aTub),

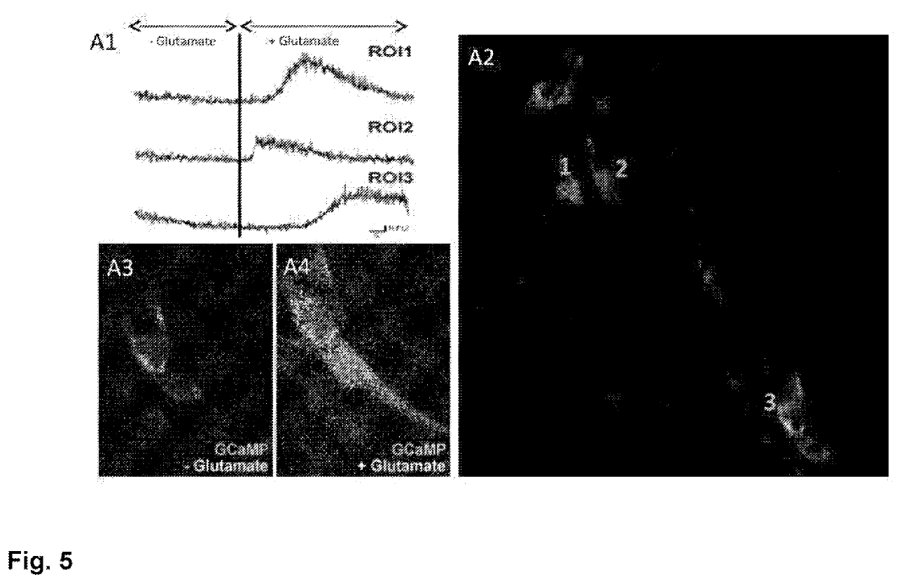

FIG. 5: shows a measurement of the total fluorescence intensities before and after the addition of the neurotransmitter glutamate,

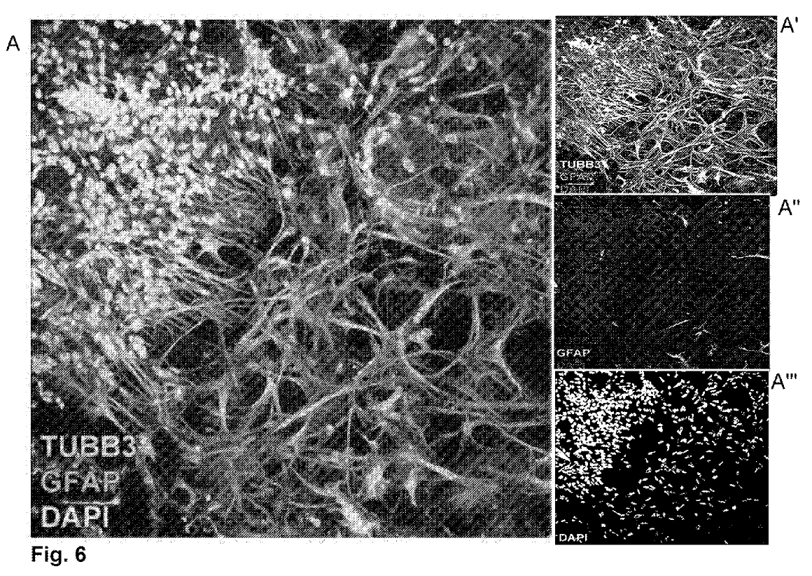

FIG. 6: show the triple staining of a hydrogel containing cells at the age of three weeks using various markers/dyes,

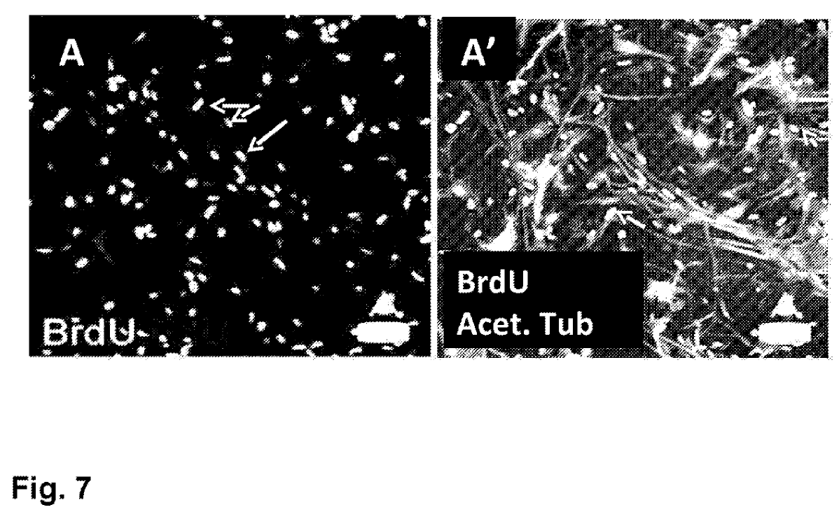

FIG. 7: shows the result of the incubation of the embedded cells with the synthetic nucleoside bromodeoxyuridine (BrdU) one week after the start of the cell culture,

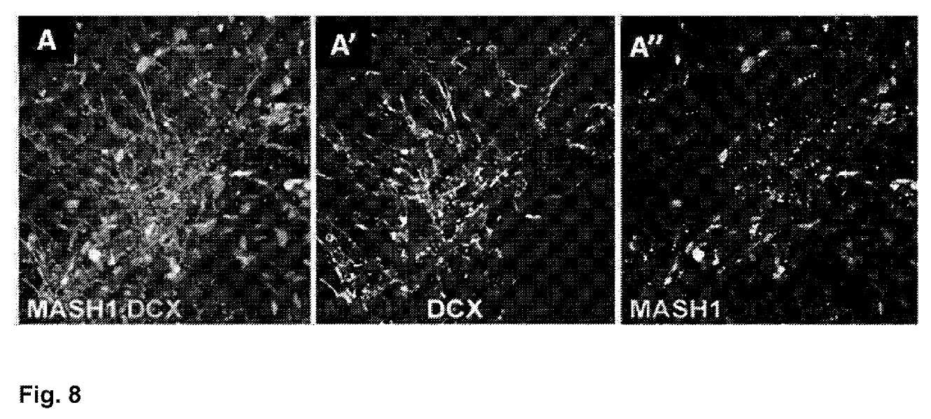

FIG. 8: shows the formation of various neuronal subtypes from newly formed neurons,

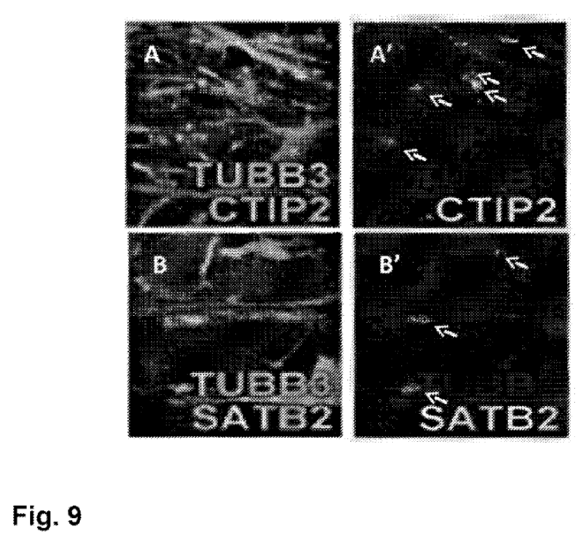

FIG. 9: shows neuronal stem and progenitor cells which are embedded in the hydrogel and are newly formed due to proliferation, after three weeks,

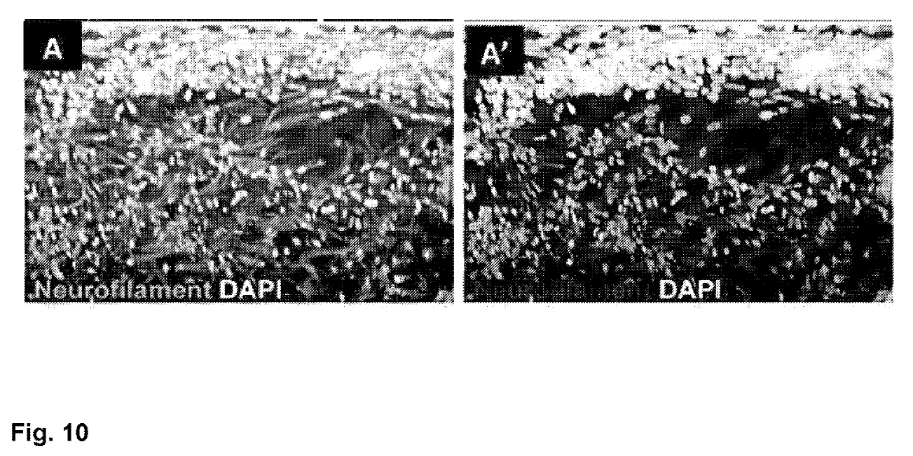

FIG. 10: shows neurofilament expression as additional evidence of the mature differentiation status of human neuronal stem and progenitor cells (NSPCs),

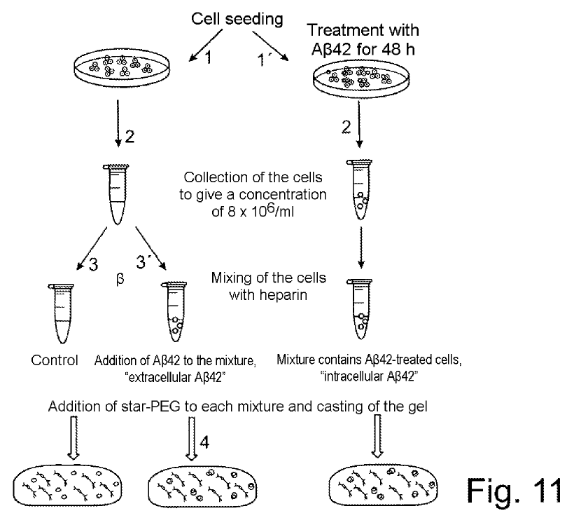

FIG. 11: shows a graphic depiction of the preparation of the PEG-heparin hydrogel for the investigation of the effect of amyloid β 42 peptides in primary human cortical cells (PHCCs),

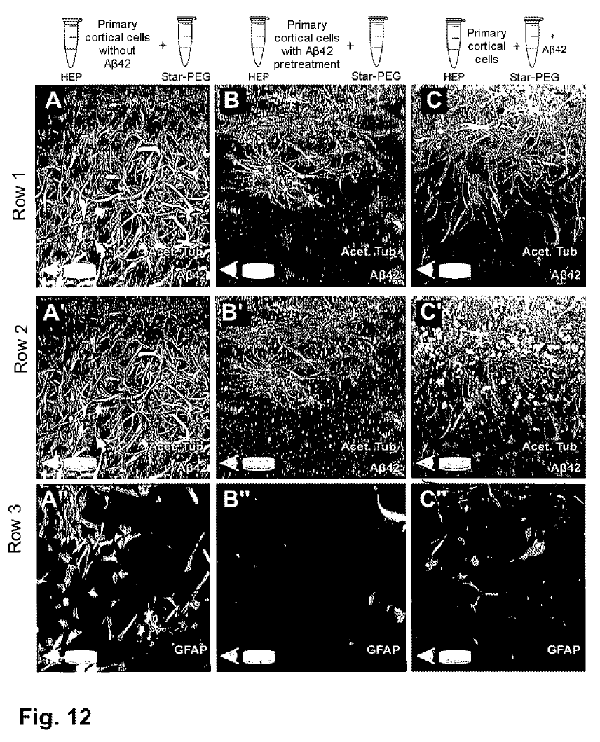

FIG. 12: shows the triple immunostaining of the hydrogel with antibodies against acetylated tubulin as neuronal cytoplasmic marker protein, with Aβ42 as marker for peptide aggregation and with GFAP as cytoplasmic marker for glial cells,

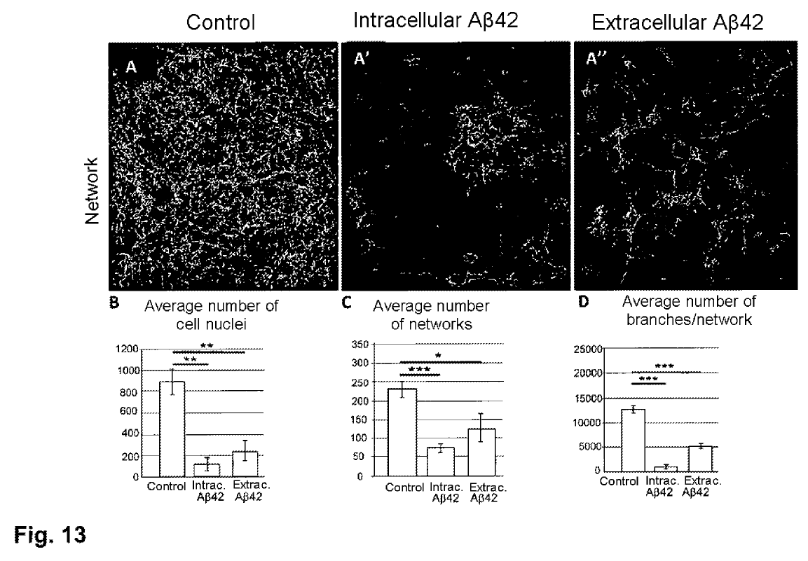

FIG. 13: shows maximum intensity projection of the skeletonized connected neuronal paths in hydrogels without Aβ42 (A), with intracellular Aβ42 (A′) and with extracellular Aβ42 (A″),

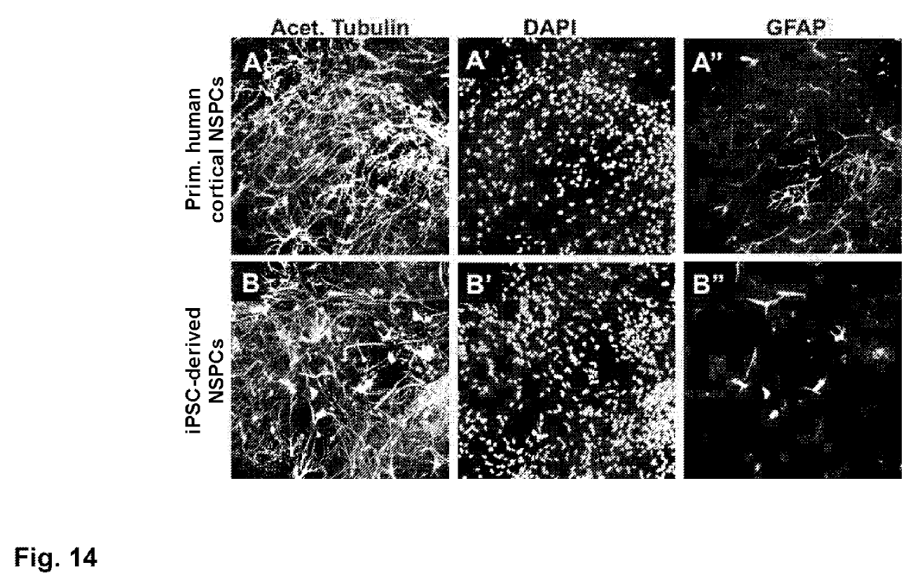

FIG. 14: shows double immunostaining against acetylated tubulin (Acet. Tubulin, image A/B) and GFAP (image A″/B″) with antibodies and nuclear staining (DAPI, image A′/B′) of human neuronal stem and progenitor cells (NSPCs) in hydrogels containing derived from (A) induced pluripotent stem cells (iPSCs) and (B) primary human cortical cells (PHCCs),

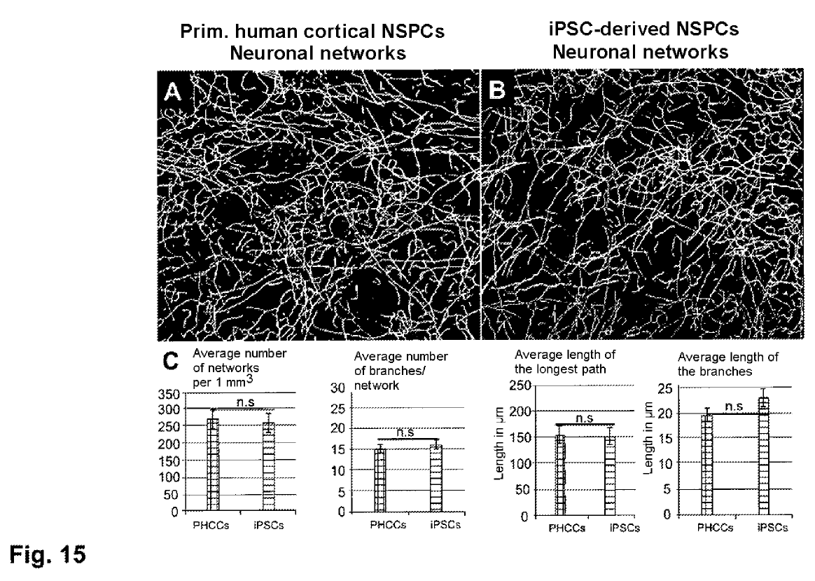

FIG. 15: shows a comparison of the maximum intensity projection of the neuronal processes of human neuronal stem and progenitor cells (NSPCs) derived from iPSCs (A) or PHCCs (B) by means of micrographs and as quantitative evaluation,

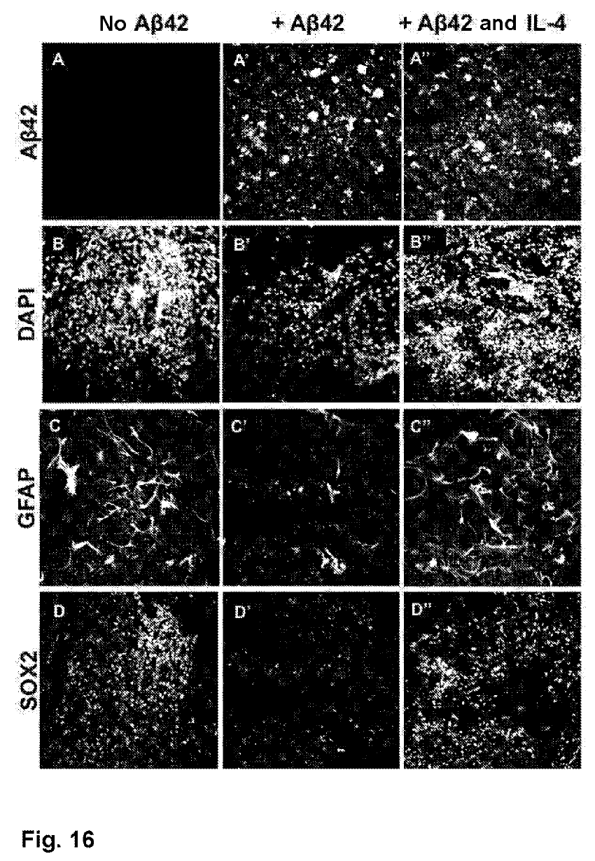

FIG. 16: shows comparative micrographs of star-PEG-HEP gels and PHCCs embedded therein for the investigation of the effect of interleukin 4 on Aβ42 toxicity,

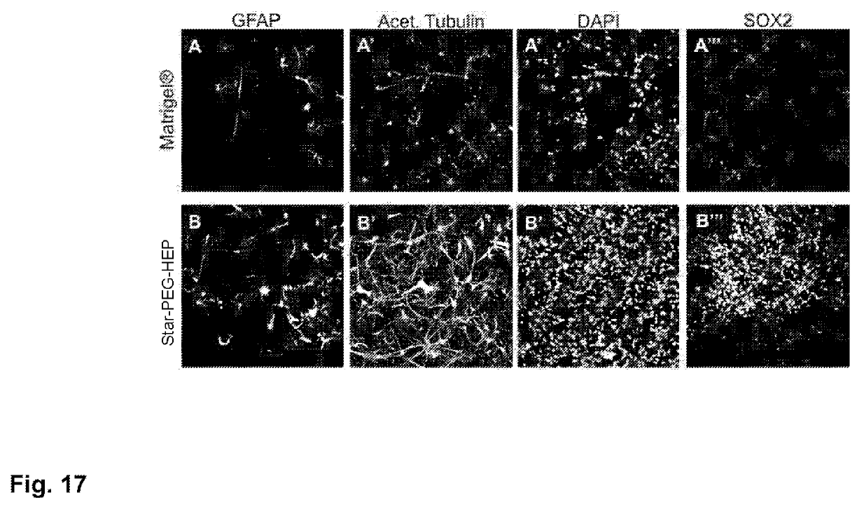

FIG. 17: shows micrographs of Matrigel and star-PEG-heparin hydrogels containing embedded PHCCs for a comparison of the glial cell population (GFAP), of the neuronal network formation (Acet. Tubulin) and of the stem-cell populations (SOX2) and of the neuroplastic capacity.

FIG. 1 shows a schematic graphic depiction of one exemplary embodiment for the preparation of a hydrogel. According to said depiction, primary human cortical cells (PHCCs) are used. They are first brought together with heparin in phosphate-buffered saline solution (PBS). As the further component of the hydrogel besides the heparin, what is used in said depiction is a conjugate of a four-arm polyethylene glycol (star-PEG) and an enzymatically cleavable peptide, with the PEG being conjugated at each arm with a peptide molecule. Said component is brought together with the heparin and the cells in phosphate-buffered saline solution (PBS). The hydrogel matrix of the hydrogel is formed by a covalent crosslinking of the thiol-terminated (cysteine side chain, cys for short) star-PEG-peptide conjugate and of a maleimide-functionalized heparin, with the hydrogel matrix being crosslinked via a Michael addition.

Several of the following figures each show microscopic images, on which it is possible to identify in each case a symbol on the bottom left or right. Said symbol is an eye which is looking at a cylindrical hydrogel either from above or from the side.

The eye looking from above means that the image labeled thereby is an image of the maximum intensity projection of a series of images on the z-axis.

The eye looking from the side means that the image labeled thereby is an image of the maximum intensity projection of a series of images on the x-axis.

FIG. 2 depicts the maturation of the neuronal network over a period of three weeks. Staining of the cytoplasmic glial cell marker GFAP (derived from the name “glial fibrillary acidic protein”) labels the cytoplasm of glial cells, which cytoplasm revealed an increase compared to the neurons. The cytoplasm of neurons is stained by means of acetyiated tubulin (aTub). Images A-A″ each show a typical image of the maximum intensity projection across the z-axis of the state of the embedded cells after one week of embedding. Images B-B″ each show a typical image of the maximum intensity projection across the z-axis of the state of the embedded cells after 2 weeks of embedding. Images C-C″ each show a typical image of the maximum intensity projection across the z-axis of the state of the embedded cell after three weeks of embedding. In the first row, what can be seen is the combinational staining of GFAP and aTub-positive cells. What can be seen in the second and third row is that the cells react positively in each case to the markers aTub and GFAP.

FIG. 3 shows an extensive three-dimensional depiction of a three-week-old gel containing neuronal and glial networks of high density. The glial cells, which are stained by means of GFAP, interact closely with neurons, which are stained by means of acetylated tubulin (aTub), a phenomenon which occurs in In vivo situations. The cell nucleus dye 4′,6-diamidino-2-phenylindole, abbreviated DAPI, labels the double-stranded nuclear DNA. DAPI-labeled cells were live at the time of fixing of the samples for evaluation. Image A in FIG. 3 shows a comprehensive network of neurons which are doubly positive with respect to aTub and DAPI. Image B shows a comprehensive network of glial cells which are doubly positive with respect to GFAP and DAPI.

FIG. 4 shows a distinctly concentrated immunoreactivity in cell junctions and synaptic boutons of embedded neurons, as also occurs in vivo in functional neurons. In this connection, images A-A′ and B-B′ show: cells doubly stained by means of acetylated tubulin (aTub) and synaptophysin (Syn). DAPI stains the cell nuclei. As shown by image A, aTub stains the processes of neurons 1 and 2, the neurons being highlighted by means of the arrows, while the circle indicates the junction of neurons 1 and 2. In image A′, the synaptophysin (Syn) staining appears as a plurality of synaptic points at the connections of neurons 1 and 2, which are labeled in image A. Image B shows a high magnification of a neuronal process which abuts a synaptic bouton. Image B′ shows how the synaptic bouton of image B reacts positively to synaptophysin (Syn).

FIG. 5 depicts the results of the measurement of the total fluorescence intensities in the case of addition of the neurotransmitter glutamate. In this connection, image A1 shows the measurement of the total fluorescence intensities before (−Glutamate) and after (+Glutamate) the addition of the neurotransmitter glutamate, as were emitted by cells 1, 2 and 3 of image A (A2). Cells 1, 2, 3 of image A2 were transfected with the calcium sensor Gcampf6, which generates an intense fluorescence signal when the cells exhibit an intracellular calcium influx as a response to the added glutamate. For this reason, an increased fluorescence signal can be observed on the graphs of A1, meaning that the transfected cells react in the presence of neurotransmitters, as also occurs in the in vivo situation. The cells in image A2 are embedded in a PEG-heparin hydrogel system before the addition of the neurotransmitter glutamate. Images A3 and A4 show a high magnification of a Gcampf6-transfected cell which is embedded in the PEG-heparin hydrogel system. Image A3 shows the cell before the addition, of glutamate. Image A4 shows that, after the addition of glutamate, an intense signal is measured owing to the influx of calcium ions.

FIG. 6 shows the triple staining of a hydrogel containing cells at the age of three weeks, having immunoreactivity with respect to the neuronal cytoplasmic marker β-III-tubulin (TUBB3), the cytoplasmic glial cell marker GFAP and the DNA dye 4′,6-diamidino-2-phenylindole (DAPI). It is possible to identify various morphological properties of the clustered cells: distinctly oriented cell processes in the image, top left, and a latticed neuronal network mixed with glial cells in the center left. Images A′-A′″ show individual images of the optical channels for TUBB3, GFAP, DAPI.

One week after the start of the culture of the cells embedded in the PEG-heparin gel, said cells were incubated with the synthetic nucleoside bromodeoxyuridine (BrdU) for 3 h. The result can be seen in FIG. 7, with arrows each pointing to stained cells. Image A shows cells stained with anti-BrdU antibody (staining of the cell nucleus). Image A′ shows that cells stained by means of anti-BrdU antibody are also positive for the cytoplasmic neuronal marker protein acetylated tubulin (aTub). FIG. 7 shows, with the aid of BrdU staining, that the cells embedded in the hydrogel proliferate and have a neuronal identity (Acet. Tub. stained cells).

FIG. 8 shows that various neuronal subtypes are formed from the neurons which are embedded in the hydrogel and are newly formed therein, which subtypes resemble those cell types which are also formed in vivo in the course of neuronal cell differentiation. In this connection, image A shows that cells in the hydrogel system are doubly positive for the neuronal progenitor cell markers MASH1, also known under the name “Achaete-scute family bHLH transcription factor 1”, ASCL1, and doublecortin/DCX. Images A′ and A″ show the individual optical channels for DCX and MASH1 staining.

FIG. 9 shows that neuronal stem and progenitor cells which are embedded in the hydrogel and are newly formed due to proliferation mature within three weeks to form cells which express marker proteins for mature cortical neurons, for example CTIP2 and SATB2.

In this connection, image A shows that the cells in the hydrogel system are doubly positive for the nuclear cortical marker CTIP2, also known under the name “B-cell CLL/lymphoma 11B”, BCL11b, and for the neuronal cytoplasmic marker TUBB3.

In this connection, arrows in image A′ point to CTIP2-positive cell nuclei of the TUBB3-positive neurons. Image B shows that the cells are doubly positive for the nuclear cortical marker SATB2, its name being an abbreviation of the name “Special AT-rich sequence-binding protein 2”, and the neuronal cytoplasmic marker TUBB3. In image B′, arrows point to SATB2-positive cell nuclei of the TUBB3-positive neurons.

FIG. 10 reveals that cells are doubly positive for the cell nucleus dye DAPI and the cytoplasmic neuronal protein neurofilament, which is expressed in mature neurons. In this connection, image A′ shows an optical channel for DAPI staining from image A. The expression of neurofilament is additional evidence of the mature differentiation status of the human neuronal stem and progenitor cells (NSPCs) after their embedding and maturation in the PEG-heparin hydrogel.

FIG. 11 contains a graphic depiction of the preparation of the PEG-heparin hydrogel for the investigation of the effect of amyloid β 42 peptides (Aβ42) in primary human cortical cells. Cells of the second passage were placed into a Petri dish in a density of 5×10³ per cm² in step 1. For the purposes of the Aβ42 neurotoxicity model, cells of the second passage were likewise placed in a Petri dish in a density of 5×10³/cm² and incubated for 48 hours with 2 μM Aβ42 (step 1′).

After 48 hours, the cells were harvested and resuspended in phosphate-buffered saline solution (PBS) at a concentration of 8×10⁶ cells per ml in a step 2. Then, the same volume of heparin solution (45 μg/μl in PBS) was added and the two were mixed to give a final concentration of 4×10⁶ cells/ml in a step 3. In the case of the coating of the extracellular environment of the cells embedded in the PEG-heparin hydrogel system with A1342, a mixture of Aβ42 peptide in a concentration of 40 μM was added in step 3′.

To cast a gel, the cell solution, PBS and heparin in step 3 were mixed with the same volume of star-PEG. In this stage, the gels cast according to step 3′ had a concentration of extracellular Aβ42 of 20 The concentration of the cells in all hydrogels is 2×10⁶ cells/ml. The reactions for gel formation last two minutes. After the casting, the gels were placed into 24-well culture plates, with each well containing a culture medium. To culture and to incubate the gels, a ratio of 5% CO₂/95% air at 37° C. was used. Gels can be cultured until the desired time point.

FIG. 12 shows the triple immunostaining of the hydrogel with antibodies against acetylated tubulin as neuronal cytoplasmic marker protein, with Aβ42 as marker for peptide aggregation and with GFAP as cytoplasmic marker for glial cells. FIG. 12 shows images of the maximum intensity projection across the y-axis of three-week-old gels without Aβ42 in images A-A″, with intracellular Aβ42 in images B-B″, with extracellular Aβ2 in images C-C″. The first row of images, i.e., images A-C, depicts the channel for the staining by means of acetylated tubulin, which highlights the neuronal networks formed. The second row of images (A′-C′) depicts the neuronal network from row 1 as well as the Aβ42 amyloid aggregates formed, which have spread within the entire volume of the gel. What is informative is the loss of neuronal networks in the presence of Aβ42 aggregates, see images B′ and C′, in comparison with the sample containing no Aβ42 aggregates, cf. figure A′. Row 3 depicts the channel for GFAP staining. The quantification of the cellular loss and of the loss of the neuronal network due to Aβ42 aggregates is depicted in FIG. 13 which follows.

FIG. 13 shows the maximum intensity projection of the skeletonized connected neuronal paths in gels without Aβ42 (A), with intracellular Aβ42 (A′) and with extracellular Aβ42 (A″).

Images A-A″ show, in all cases, the channel for aTub-positive cells, i.e., in the case of the control without Aβ42 (A), with intracellular Aβ42 (A′) and with extracellular Aβ42 (A″) of FIG. 12. Image B of FIG. 13 shows the quantification of the average cell count in gels without Aβ42, with intracellular Aβ42, with extracellular Aβ42. Image C shows the quantification of the average number of networks in gels without Aβ42, with intracellular Aβ42, with extracellular Aβ42. Image D shows the quantification of the average number of branches per network in hydrogels without Aβ42, with intracellular Aβ42 and with extracellular Aβ42.

A culture condition in which neuronal networks are formed by human neural stem and progenitor cells (NSPCs) was created by generating three-dimensional PEG-heparin hydrogels containing MMP-cleavable sites. This modification allows the cells to restructure their environment. The hydrogel synthesis is described in Tsurkan M. V. et al. Defined Polymer-Peptide Conjugates to Form Cell-Instructive starPEG-Heparin Matrices In Situ. Advanced Materials (2013).

When cells are introduced into said PEG-heparin hydrogels during the polymerization stage, it is observed surprisingly that, just one week after introduction of the cells, the gel contains sparsely distributed GFAP-positive glial cells with a 3D-branched morphology. After two weeks of the cell culture, the spread and arrangement of neurons positive for acetylated tubulin is observed in clusters. After three weeks of culturing, the cell cultures show extensive, complex networks of neurons with interspersed glial cells. Moreover, the 3D cultures of the NSPCs are stainable by means of the synaptic marker synaptophysin, which accumulates at the neuronal nodes and nodal points, indicating more mature synaptic connections in comparison with 2D cultures. By contrast, in 2D cultures with neuronal and glial cells, it is not possible to observe synaptophysin staining in clusters of synapses.

Neurons in 3D hydrogels are also responsive to neurotransmitters, such as glutamate, and this can be demonstrated by the increase in the intracellular calcium level, which is determined by means of the transfection of GCamP6f-expressing plasmids containing a CMV promoter-driven calcium sensor. These results show that 3D cultures of the NSPCs are capable of generating a comprehensive network of neurons and glial cells in a three-dimensional arrangement. Older cortical subtype markers such as CTIP2 and SATB2, proneural markers Mash1 and DCX and the mature neuronal marker neurofilament (marker protein for differentiated neurons) are also expressed in NSPC cultures, indicating that the neuronal cells present in the 3D cultures develop under the prevailing conditions to form mature neurons.

Amyloid β 42 peptide, a misfolded protein relevant to Alzheimer's disease, was also used in order to model its toxicity and its effects on neuronal networks. The method according to the invention using a three-dimensional hydrogel system for culturing can, in this way, also be used as a model for neurodegenerative diseases. It has been possible to show that amyloid beta 42 accumulation impairs neuronal network formation and neuronal connectivity in vivo and in vitro. To investigate whether amyloid β 42 (Aβ42) influences the neuronal network, cultures were generated by treating the cells with Aβ42 before introduction into the hydrogel system (intracellular Aβ42) or incubating the hydrogels with Aβ42 before introduction of the cells (extracellular Aβ42). In comparison to the control gels, in which it was possible to observe the formation of extensive network forms, there was significant impairment of network formation by intracellular and extracellular Aβ42. In comparison with the control gels, Aβ42-treated gels contain a significantly reduced number of cells and networks. In addition, it can be observed that, even if some networks are formed in Aβ42-treated gels, said networks contain a significantly lower number of branches per network. Moreover, it was found that the Aβ42 treatment led, as in the human brain, to dystrophy of axons. These results show that the 3D gels can recapitulate the human pathophysiology of Aβ42, which exerts a toxic effect on the formation of neuronal networks, irrespective of the neurogenic capacity of the neuronal stem or progenitor cells. Furthermore, the three-dimensional hydrogel system can be used as a practical screening platform for testing compounds which might restore the neurogenic capacity of human stem cells and the formation of neuronal networks even in the presence of Aβ42.

In a further development of the prior art, a modular and easily controllable hydrogel material system is used which makes it possible to modulate independently cell-instructive signals which occur in the natural cell environment (the so-called extracellular matrix (ECM)), but especially the physical network properties (stiffness of the hydrogel within a range from 200 Pa up to 6 kPa), the degradability and the biomolecular composition (functionalization with adhesion and signaling peptides, soluble cytokines and growth factors), as known from Tsurkan M. V. et al. Defined Polymer-Peptide Conjugates to Form Cell-Instructive starPEG-Heparin Matrices In Situ. Advanced Materials (2013). Therefore, it is possible to test a multiplicity of parameters systematically (and independently of one another) in a range of material properties. Furthermore, the hydrogel is crosslinked under mild, cell-friendly conditions to allow a high viability of the cells. Furthermore, the cells can be printed within the matrix with a zonal heterogeneity in order to generate zonally differentiated structures having a good viability.

The three-dimensional cultures used contain neuronal cells which are positive for marker proteins of mature neurons, such as, for example, CTIP2 and SATB2. This is an indication of mature cortical neurons which are formed in culture and which exhibit a degree of cell differentiation in a manner highly similar to in vivo conditions. Furthermore, it was possible to measure the electrophysiological activity and membrane-channel activity in cultured cells in 3D, a function which likewise shows the in vivo-type characteristics of the system used according to the invention.

Cultures containing extensive neuronal networks can be generated in three weeks and can survive for at least 10 weeks. Owing to the rapid generation and the culture conditions, it is possible to use hydrogel 3D cultures for iPS-based personalized medicine during any brain disease in order, for example, to test the effects of various active ingredients on patient cells prior to a clinical treatment. Furthermore, the method according to the invention facilitates the rapid expansion of glial and neuronal progenitor populations and can be used for cell-based therapies, which require a large number of cells.

A further major advantage of the 3D hydrogel cell culture system described here for the first time is the transparency of the gel material which encloses the cells. For analytical purposes, the 3D cultures are transparent and can be used for microscopic real-time recordings and other analyses which require a good transparency of the tissue. Accordingly, the 3D cultures allow quantitative measurements of network formation and neuronal branches via optical and microscopic methods, it being possible to observe network formation over the entire culturing period. As already mentioned, the system also allows the measurement of electrophysiological activity and of the membrane-channel activity of the individual neurons and of the neuronal circuits. Furthermore, an algorithm was developed in order to follow the cellular connections in the gels and to describe the statistical results quantitatively.

On the basis of currently available information, the star-PEG-heparin culture system containing primary human cortical cells that is preferably used is the only 3D culture system for neuronal cells which provides quantifiable and comprehensive neuronal networks.

In the system from Kim et al., the neuronal network is of distinctly lower quality in comparison with the much larger neuron network which is obtainable by means of the method according to the invention and which extends over the entire culture space provided by the PEG-heparin hydrogel used according to the invention. In the system according to Koutsopoulos S. et al., a low cell survivability was found and the quality of the network is also distinctly worse than that provided by the cell-responsive star-PEG-heparin system.

Moreover, the two systems (Kim/Koutsopoulos et al.) are based on BD Matrigel, an extracellular matrix extracted from the Engelbreth-Holm-Swarm (EHS) mouse sarcoma, a tumor tissue. In addition to a high batch-to-batch variation which is known for Matrigel and which complicates the reproducibility of results, the tumor-typical signaling substances and cell components which are contained by such a product alter the normal molecular signal transmission to the cells. Therefore, such a product is not suitable for investigations in relation to brain development and not suitable for transplantation and/or investigations of neurodegenerative processes, since the molecular processes in vivo differ greatly from the environment present in the tumor tissue. The hydrogel system used according to the invention is based on a synthetic PEG and biologically derivatized, but purified heparin having well-known molecular properties such as molecular weight distribution and functionality, which are always tested before use. Therefore, the present system also exhibits no problems with reproducibility and also no immunogenic reactions.

Moreover, Dawai Zhang and Koutsopoulus et al. used a self-organizing peptide hydrogel and a type I collagen gel for the culturing of neuronal cells. In the case of collagen, there are—just as in the case of Matrigel—variations in different batches. In the case of all other hitherto used hydrogel systems (e.g., PuraMAtrix, self-organizing peptides, collagen I and Matrigel), the physical properties are rather undefined and highly variable and cannot be varied independently of the biomolecular composition. Accordingly, the materials used in this connection do not allow independent investigation of the influence of mechanical and biomolecular stimuli and suffer from poor reproducibility.

Since these various stimuli which arise from the composition and arrangement of the extracellular matrix (ECM) are a major parameter for the influencing of stem-cell activity and for new tissue formation, the systems known from the prior art do not make it possible to investigate mechanical and biomolecular signals separately from one another. On the contrary, in vitro assays are, owing to the complex interaction among the multiplicity of extracellular matrix-derived signals and their pleiotropic effects, a challenge in the identification of the function of exogenous stimuli on tissue structuring.

As such, the well-defined and modularly tailorable PEG-heparin hydrogels used here can be used for modulating the mechanical and biomolecular signals independently of one another, since it is possible to set the composition of the hydrogels independently (Tsurkan M. V. et al. Defined Polymer-Peptide Conjugates to Form Cell-Instructive starPEG-Heparin Matrices In Situ. Advanced Materials (2013)). Furthermore, the 3D hydrogel systems according to the invention are also cell-responsive, for example by means of MMP-cleavable sites, and this allows, for example, cell-triggered reconstruction processes and substitution by their own matrix in order to achieve a greater similarity with the in vivo conditions in brain tissue. The previously described known methods for three-dimensional neuronal networks lack a defined composition which allows the specific modulation of the mechanical and biomolecular properties of the 3D culture system as well as the cell-dependent reorganization of the extracellular matrix in the 3D hydrogel.

Therefore, the 3D cell culture platforms used could serve as an advantageous cell culture system for clarifying the role of matrix properties in stem-cell activity and differentiation, provided that the cells interact dynamically with the hydrogel system in order to generate a cell-covered extracellular matrix. In addition, the 3D hydrogel systems used can be coated either covalently with adhesion or signaling molecules or noncovalently with heparin-binding signaling molecules. Overall, the influence of multiple cell-instructive exogenous signals can be investigated systematically with regard to the cell proliferation of human neuronal stem and progenitor cells and with regard to neuronal network formation.

Many 3D systems, including organoids, cannot form structures which are reproducible in size and shape. The 3D cultures according to the invention can be adjusted and specifically controlled for these two parameters and thus offer substantially better defined conditions for the 3D cell culture.

The biodegradable hydrogel which was described in U.S. Pat. Nos. 6,306,922 A and 6,602,975 A is a photopolymerized hydrogel. This means that, for the polymerization and gel formation under specific electromagnetic conditions, said hydrogel requires a special instrument which emits ultraviolet light (UV light). UV light leads to the generation of free radicals at the embedded cells and to the induction of apoptotic signaling pathways, which may lead to cell death and which adversely affect cell viability. Moreover, UV light causes DNA mutations and damage to the cellular DNA of the embedded cells. By contrast, in the innovative 3D hydrogel system described here, the matrix is polymerized at room temperature and without the use of UV light. In this way, DNA damage and mutations to the cells do not arise, and so there is better maintenance of cellular functions. In addition, dispensing with a UV-induced polymerization allows the use of the 3D hydrogel system without the use of expensive instruments. This advantage makes the system user-friendly and thus ideal for use outside highly specialized laboratories.

Furthermore, the present system is the only one which, with use of plasmids, allows specific gene misexpression in order to overexpress a functional version of a gene (enhancement of function) or to downregulate a gene, for example by use of siRNAs (small interfering RNAs) or of nonfunctional dominant-negative variants of a gene (attenuation or loss of function).

The use of a calcium sensor (GcaMP) driven by a plasmid expression system was described. The misexpression system is based on the plasmid transfection method tailored to the 3D gels.

Compared to earlier reports on the modeling of Alzheimer's disease (AD) in 3D cultures using Matrigel, PEG-heparin 3D gels allow a significantly more rapid development of networks, which, for example, provides an advantage for use in high-throughput screening platforms for active ingredients.

To date, it has not been possible to show comprehensive network formation in 3D gel systems with human neurons. Previous 3D cultures used floating or Matrigel-embedded systems and use modified cells.

In earlier 3D cultures, it was not possible to examine the quality of network formation, since it was not possible to quantify the formation of networks. The three-dimensional cultures obtainable by means of the method according to the invention allow quantitative measurements of network formation, for example the length and number of axons and neurites, number of branching and linking points, and of neuronal branching. This was hitherto not possible. Furthermore, systems used according to the invention allow real-time recordings and the monitoring of the embedded cells during the cell culture period.

The 3D cultures for the mature cortical neurons express cortical markers, such as, for example, CTIP2 and SATB2.

In the cultured cells, it was possible to measure, in 3D, electrophysiological activity and membrane-channel activity by utilizing the transfection of genes with use of plasmid vectors. In the case of earlier studies with 3D cultures in scaffolds or as organoids, it was necessary to manipulate the genome of the cells. With the possible transfection method, it is possible to carry out a misexpression without genetic modification of the cells themselves or the use of viruses.

Cultures containing expanded neuronal networks can be generated in 3 weeks and can be kept alive for more than 16 weeks. Owing to the rapid generation and the culture conditions, it is possible to also use the 3D cultures for iPS-based personalized medicine during any brain disease in order to test the effects of various active ingredients on patients, on the patient's own cells, prior to a clinical treatment.

The cultures are optically transparent and can be used for real-time recordings and other analyses which require a clear visibility of the tissue. This is not the case for previously known 3D-scaffold-based or organoid-based systems.

There is no need to genetically modify the cells in order to form 3D networks.

The method according to the invention using a hydrogel system allows the most rapid expansion of glial cells and neuronal progenitor cell populations and can be used for cell-based therapies in which large quantities of cells are required.

### TECHNICAL DETAILS

### Example 1

In Example 1, primary human cortical cells (PHCCs) were used.

The PHCCs were isolated from the cerebral cortex from donated tissue from fetuses from the 21st week of pregnancy, and were purchased in a frozen state in the first passage from ScienCell Research Laboratory (SRL, catalog number 1800). The cells were certified as negative with regard to HIV-1, HBV, HCV, mycoplasma, bacteria, yeast and fungi. The PHCCs were placed into conventional T75 flasks or 24-well plates and cultured at 37° C. in an incubator having an atmosphere of 5% CO₂195% air using astrocyte medium (SRL, catalog number 1801) supplemented with 5% fetal bovine serum (SRL, catalog number 0010), 1% of astrocyte growth agent (SRL, catalog number 1852) and 1% of penicillin/streptomycin solution (SRL, catalog number 0503).

PEG-heparin hydrogels were prepared as described in Tsurkan et al., Advanced Materials 2013, vol. 25 (18) pp. 2606-2610, with the following changes: PHCCs were collected from culture vessels using Accutase® (from Invitrogen) as cell-detachment medium. After centrifugation for 10 min at 12 000 revolutions per minute, the cells were resuspended in phosphate-buffered saline solution (PBS) at a concentration of 8×10⁶ cells per ml. The polymeric starting materials (precursors) for the hydrogel preparation consisted, as described in Tsurkan et al., Advanced Materials 2013, vol. 25 (18) pp. 2606-2610, of heparin functionalized with six maleimide groups (HEP-HM6) having a molecular weight of 15 000 g/mol, and four-arm starPEG functionalized with enzymatically cleavable peptide sequences at each arm having a total molar mass of 15 500 g/mol (starPEG-MMP). The hydrogels were formed by mixing the starting materials in a molar ratio of 0.75 mol of starPEG-MMP to 1 mol of HEP-HM6, corresponding to a degree of crosslinking of 0.75, at a total solids content of 3.9%. To this end, for each hydrogel, the cells were first resuspended in 5 microliters (pi) of PBS, then 5 μl of HEP-HM6 solution (0.448 mg of HEP-HM6 dissolved in 5 μl of PBS) and 10 μl of the starPEG-MMP solution (0.347 mg of starPEG-MMP dissolved in 10 μl of PBS) were added, as described in Tsurkan et al., Advanced Materials 2013, vol. 25 (18) pp. 2606-2610, mixed intensively within a few seconds, and thus a final volume of 20 μl of hydrogel having a concentration of cells of 2×10⁶ cells/ml was generated. The 20 μl drops were immediately subsequently applied to a Parafilm sheet, followed by waiting for a further two minutes until gel formation was completed. The gels were then placed into 24-well culture plates, with each well containing one 20 μl-drop hydrogel and 1 ml of culture-medium volume. The hydrogels were then cultured in the wells at 37° C. under 5% CO2/95% air until the desired time point. After gel formation, the resulting hydrogels had a storage modulus within a range of 450±150 Pa, which was determined by means of oscillatory rheometry of hydrogel slices swollen in PBS at room temperature by using a rotational rheometer (ARES LN2; TA Instruments, Eschborn, Germany) having a plate-plate measurement arrangement at a plate diameter of 25 mm through frequency-dependent measurement at 25° C. within a shear frequency range of 10⁻¹-10² rad s⁻¹ with a deformation amplitude of 2%.

### Example 2

Formation of PEG-heparin and embedding of cells:

The PEG-heparin gels were prepared as described in Tsurkan et al., Advanced Materials 2013, vol. 25 (18) pp. 2606-2610, with the following changes:

PHCCs of the second passage were collected from culture vessels the culture vessel using Accutase® (from Invitrogen) as cell-detachment medium. After centrifugation for 10 min at 12 000 revolutions per minute, the PHCCs were resuspended in phosphate-buffered saline solution (PBS) at a concentration of 8×10⁶ cells per ml. The polymeric starting materials for the hydrogel preparation consisted, as described in Tsurkan et al., Advanced Materials 2013, vol. 25 (18) pp. 2606-2610, of heparin functionalized with six maleimide groups (HEP-HM6) having a molecular weight of 15 000 g/mol, and four-arm starPEG functionalized with enzymatically cleavable peptide sequences at each arm having a total molar mass of 15 500 g/mol (starPEG-MMP). The hydrogels were formed by mixing the starting materials in a molar ratio of 0.75 mol of starPEG-MMP to 1 mol of HEP-HM6 (corresponds to a degree of crosslinking of 0.75) at a total solids content of 3.9%. To this end, for each hydrogel with a total volume of 20 μl, the cells were first resuspended in 5 microliters (pi) of PBS, then 5 μl of HEP-HM6 solution (0.448 mg of HEP-HM6 dissolved in 5 μl of PBS) and 10 μl of the starPEG-MMP solution (0.347 mg of starPEG-MMP dissolved in 10 μl of PBS) were added, as described in Tsurkan et al., Advanced Materials 2013, vol. 25 (18) pp. 2606-2610, mixed intensively within a few seconds, and thus a final volume of 20 μl of hydrogel having a concentration of cells of 2×10⁶ cells/ml was generated. The 20 μl drops were immediately subsequently applied to a Parafilm sheet, followed by waiting for a further 2 minutes until gel formation was completed. The gels were then placed into 24-well culture plates, with each well containing one 20 μl-drop hydrogel and 1 ml of culture-medium volume. The culture conditions used were 5% CO2/95% air at 37° C. The hydrogels were then cultured in the wells until the desired time point. After gel formation, the resulting hydrogels had a storage modulus within a range of 450±150 Pa, which was determined by means of oscillatory rheometry of hydrogel slices swollen in PBS at room temperature by using a rotational rheometer (ARES LN2; TA Instruments, Eschborn, Germany) having a plate-plate measurement arrangement at a plate diameter of 25 mm through frequency-dependent measurement at 25° C. within a shear frequency range of 10⁻¹-10² rad s⁻¹ with a deformation amplitude of 2%.

To use gels which were pretreated with amyloid β 42 (Aβ42), the cells were incubated with 2 μM Aβ42 for 48 hours prior to the cell collection from the culture vessel and prior to the embedding of the cells in the hydrogel. To generate a gel environment which contains Aβ42, the cells were first dissolved in 4 μl of 100 μM Aβ42 peptide in PBS. 6 μl of the heparin solution (0.448 mg of HEP-HM6 dissolved in 6 μl of PBS) and 10 μl of the starPEG-MMP solution (0.347 mg of starPEG-MMP dissolved in 10 μl of PBS) were added and, as described above, mixed. In this gel mixture, the concentration of Aβ42 is 20 μM and the concentration of the cells is 2×10⁶ cells per ml.

### Example 3

To use gels which were pretreated with amyloid β 42 (Aβ42), the cells were incubated with 2 μM Aβ42 for 48 hours prior to the cell collection from the culture vessel and prior to the embedding of the cells in the hydrogel.

Immunocytochemistry:

All hydrogels were fixed with ice-cold paraformaldehyde and incubated at room temperature for 1.5 h, followed by a wash in PBS overnight at 4° C. For the immunocytochemistry, the hydrogels were blocked for 4 h overnight in blocking solution which consisted of 10% normal goat serum, 1% bovine serum albumin, 0.1% Triton-X in PBS. The gels were washed at 4° C. for two consecutive days with occasional change of the PBS. After washing, the gels were incubated with secondary antibody at room temperature for 6 hours (1:500 in blocking solution). After 3 wash steps of 2 hours, DAPI staining was carried out in each case (1:3000 in PBS, 2 hours at room temperature).

Fluorescence Recordings

For the hydrogels, fluorescence recordings were carried out using a Leica SP5 inverted confocal and multiphoton microscope. The hydrogels were placed into glass-bottom Petri dishes. 60 μl of PBS were added to the upper side of the hydrogels in order to prevent drying. The Z-stacks were captured using a water immersion lens (25×). Each Z-stack has a z-distance of 500 μm.

Comparison of the Development of Primary Human Cortical Cells (PHCCs) and iPSC-Derived Neuronal Stem and Progenitor Cells (NSPCs) in Star-PEG-Heparin Hydrogels

FIG. 14 shows micrographs allowing a comparison of embedded PHCCs and iPSC-derived NSPCs with respect to their capacity to form neuronal networks in star-PEG-heparin hydrogels. In this connection, images A-A″ show the maximum intensity projection of a 500 μm thick Z-stack of iPSC-derived NSPCs embedded in star-PEG-heparin hydrogels modified with RGD peptides, stained for acetylated tubulin (Acet. Tubulin, see image A), stained with DAPI (image A′) and stained by means of GFAP (image A″). Images. B-B″ show the maximum intensity projection of a 500 μm thick Z-stack of PHCCs embedded in star-PEG-heparin hydrogels, stained for acetylated tubulin (image B), stained with DAPI (image B′) and stained with GFAP antibodies (image B″).

FIG. 15 shows, in image A, the maximum intensity projection of the neuronal processes of human cortical NSPCs after TUBB3 staining. Image B of FIG. 15 shows the maximum intensity projection of the neuronal processes of iPSC-derived NSPCs after TUBB3 staining. Image C shows the quantification and contrasting of the neuronal network properties of images A and B in graphs.

The hydrogels used according to the present invention can be covalently modified with various matrix-derived peptides such as RGD (Arg-Gly-Asp) or be used for the effective administration of soluble signaling molecules. In this way, effects of exogenous signals can be individually tested on the human neuronal stem and progenitor cell proliferation and on the neuronal network formation. The fact that this star-PEG-heparin hydrogel system can be modified with a multiplicity of different molecules provides a user with the possibility of creating customized environments. For example, the adjustment of the PEG-HEP scaffold with RGD peptides makes it possible to culture human iPSC-derived neuronal stem and progenitor cells (NSPCs), as shown in FIG. 14. Such a method is not possible without the RGD modification. As can be seen from FIG. 15, there are no differences in the hydrogel system used when comparing the capacity of, firstly, the primary human cortical cells (PHCCs) and, secondly, iPSC-derived neuronal stem and progenitor cells (NSPCs) to form neuronal networks. The number of networks and the number and length of branches is comparable, as illustrated by especially image C in FIG. 15. The highly similar development of human iPSC-derived neuronal stem and progenitor cells (NSPCs) compared to those from primary human cortical cells (PHCCs) shows that the abovementioned hydrogel system can be used in a broad spectrum of uses. The most promising uses of the hydrogel system are to be expected in the field of personalized medicine. This is suggested by, in particular, the modifiability, the responsiveness to treatments, such as with interleukin 4 (IL-4) for example, and also the ability to produce large quantities of the star-PEG-heparin hydrogels within a relatively short time. By culturing iPSC-derived neurons from patients, it is possible to better understand different neuronal developmental disorders. The hydrogels generated can, however, also be used for identifying treatment strategies.

Preparation of PEG-Heparin Gels with iPSC-Derived Neural Stem and Progenitor Cells (NSPCs)

Human neuronal stem and progenitor cells (NSPCs) derived from iPSCs, named HIP™ (BC1 line), were purchased from Amsbio (catalog number: GSC-4311). These NSCs were thawed as specified by the manufacturer and cultured in Geltrex-coated cell culture flasks. For the expansion and the further culturing of the cells, use was made of the expansion medium according to the instructions from the manufacturer. NeuralX™ NSC medium has the following composition: 2% GS22™ neuronal supplement, 10; 1× nonessential amino acids, 2 mM L-alanine/L-glutamine; 20 ng/ml FGF2. The HIP™ NSCs were detached from the cell culture flasks using Accutase (Invitrogen). After centrifugation at 12 000 rpm for 10 minutes, the HIP™ NSCs were resuspended in PBS in a density of 8×10⁶ cells/ml. For each hydrogel, the cells were first resuspended in 5 microliters (μl) of PBS, then 5 μl of heparin solution (45 μg/μl in PBS) and 2 M integrin ligands as RGD peptides (Tsurkan, Chwalek et al., 2011, Maltz, Freudenberg et al., 2013, Tsurkan, Chwalek et al. 2013, Wieduwild, Tsurkan et al. 2013) dissolved in PBS by thorough vortexing and 10 μl of PEG were added to give a final volume of 20 μl containing 2×10⁶ cells/ml. A 20 μl drop was applied to a Parafilm sheet. Gel formation took two minutes. The gels were placed into 24-well culture plates, with each well containing 1 ml of HIP-expansion-medium volume. The gels were cultured by using 5% CO₂/95% air at 37° C. The gels can then be cultured until the desired time point.

Use of the PEG-HEP Hydrogels for the Analysis of the Effect of Individual Factors on Neurogenic Plasticity and Aβ42-Mediated Toxicity

FIG. 16 shows micrographs of star-PEG-HEP gels containing embedded PHCCs from the control group without Aβ42 (A-D), the control group with Aβ42 (A′-D′) and the culture with Aβ42 and interleukin 4 (IL-4) (A″-D″), in each case after staining with anti-Aβ42 antibodies (images A-A′), after staining with DAPI (images B-B″), with anti-GFAP antibodies (images C-C″), and after staining with anti-SOX2 antibodies (images D-D″).

To test whether IL-4 acts in humans in a similar manner as could be previously shown in zebrafish investigations for example, the abovementioned star-PEG-HEP hydrogels containing PHCCs were used. To this end, hydrogels containing embedded PHCCs were prepared and they were incubated with Aβ42, as already described. Each test setup contained a positive (with Aβ42) and a negative control group (without Aβ42) as well as an experimental group with Aβ42 and IL-4. In the experimental group, the star-PEG-HEP gels containing embedded PHCCs were cultured with Aβ42 and, at the same time, in the presence of 100 ng/ml IL-4 in the medium. After a three-week culturing phase, the samples were fixed and immunologically stained with respect to GFAP and SOX2 in order to investigate effects of IL-4 on the neural stem and progenitor cells. The nucleus dye DAPI was used in order to show entire cells. In Aβ42-treated cell cultures, it was possible to observe a strong decline in the cell count in comparison with untreated control cultures, with both GFAP-positive glial cells and SOX2-positive neurons being affected. In cultures treated with Aβ42 and, at the same time, with IL-4, the cell count was altogether comparable with the control cultures without Aβ42 treatment. The results indicate that a treatment with IL-4 can counteract the neurotoxic effect of Aβ42. It can be concluded that the treatment with interleukin 4 stimulates GFAP-positive cells in relation to proliferation to form more neuronal stem and progenitor cells. IL-4 thus increases the neuroplasticity of the embedded PHCCs despite the presence of neurotoxic Aβ42. Overall, the data show that the treatment with IL-4 activates the proliferation of human neuronal stem cells, just as shown in the zebrafish model. IL-4 is thus an important candidate for future therapies against Aβ42-mediated neurodegeneration. The administration of specific Aβ42 peptides having cell-penetrating sequences to the present hydrogel-based 3D cultures shows that the cell culture method used here can reproduce the pathophysiology of human Aβ42 toxicity and that neuroprotective effects can also be investigated in the star-PEG-HEP hydrogel system.

Comparison of a Conventional Method for Preparing 3D Cell Cultures with the Star-PEG-HEP Hydrogel System

FIG. 17 shows micrographs after immunostaining with respect to GFAP, SOX2 and acetylated tubulin for the comparison of Matrigel and star-PEG-heparin hydrogels, in which primary human cortical cells (PHCCs) are embedded in each case. In this connection, images A-A′″ show PHCCs embedded in Matrigel. By contrast, images B-B′″ show PHCCs embedded in star-PEG-heparin hydrogels. Images A and B are each stained for glial fibrillary acidic protein (GFAP) in order to identify the glial cell population. Images A′ and B′ are each stained with respect to acetylated tubulin (Acet. Tubulin) in order to show the neuronal network formation. Images A″ and B″ contain DAPI staining in order to label entire cells. Lastly, the opposing placement of images A′ and B′″ allows a comparison of the extent of the stem-cell populations and of the neuroplastic capacity by means of SOX2 staining.

The three-dimensional topological organization in the organism gives tissues properties such as structure, lineage specification and spatial interaction, which cannot be reproduced in conventional two-dimensional (2D) cell cultures. As a result, three-dimensional (3D) cell culture systems have a distinct advantage and are used extensively. Matrigel-based 3D cell cultures are currently the preferred standard of such techniques, with neuronal cells growing in a viscous gel material in which extracellular matrix (ECM) proteins, such as collagen and laminin, are embedded. However, Matrigel-based products are chemically undefined and heterogeneous in their composition and cannot be altered in various properties such as stiffness, scaffold composition or biological responsiveness. This complicates the interpretation of results and it is hardly possible to precisely analyze the influences of various exogenous and paracrine signals on cellular development. By contrast, the hydrogel system used according to the invention and based on heparin and PEG provides valuable advantages by allowing the independent adjustment of biophysical and biomolecular matrix signals. In a direct comparison between the Matrigel system and the system used according to the invention in FIG. 17, it can be easily observed that the cellular composition of the glial cells is very similar in both matrix systems, but the neurogenic capacity and the capacity of the human stem cells to form neuronal networks is distinctly higher in the star-PEG-heparin hydrogels than in the Matrigel matrix. At this point, it should be pointed out that both culture systems were cultured for the same period of three weeks in the same growth medium without further additives. The observance of neuronal networks in the star-PEG-heparin hydrogel, but not in the Matrigel system, is confirmed by the fact that it was hardly possible in the Matrigel system to identify mature synaptic connections between neurons. By contrast, the neurons in the star-PEG-heparin hydrogels formed neuronal networks. The reason why NSPCs in the Matrigel system do not manifest their neurogenic properties and do not form networks is presumably due to the highly unorganized ECM environment, which acts similarly to scar tissue.

Preparation of the Matrigel 3D Culture

For the Matrigel cell cultures, Matrigel from BD Biosciences (catalog number: 356234) was used. Prior to each cell culture procedure and use of Matrigel, pipette tips and Eppendorf tubes were frozen at −20° C. in accordance with the manufacturer's instructions for the “thick gel method”. The Matrigel was thawed at 4° C. overnight on ice. PHCCs of the second passage were detached from cell culture flasks using Accutase (Invitrogen). After centrifugation (at 12 000 rpm for 10 minutes), the PHCCs were resuspended in BD Matrigel in a density of 2×10⁶ cells per ml. Droplets of the cell/Matrigel mixture were generated for solidification at 37° C. Thereafter, cell culture medium (SRL, catalog number 1801) was added and the gels were cultured for three weeks. In this connection, the cell culture medium was changed on the day after the preparation of the cell/Matrigel mixture and then on every second day.

### List of Abbreviations

Aβ42 amyloid β 42
Acet.Tub acetylated tubulin
aTub acetylated tubulin
BrdU bromodeoxyuridine
CTIP2 a marker protein for mature cortical neurons, also known under the name “B-cell CLL/lymphoma 11B”, BCL11b
DAPI 4′,6-diamidino-2-phenylindole, a cell nucleus dye
GFAP glial fibrillary acidic protein, cytoplasmic marker for glial cells
Gcamp calcium sensor
HEP-HM6 heparin conjugated with six maleimide groups
IL-4 interleukin 4
iPSCs induced pluripotent stem cells
MMP matrix metalloprotease
NSPCs human neuronal stem and progenitor cells; abbreviation of the term: neural stem and progenitor cells
PBS phosphate-buffered saline solution
PEG polyethylene glycol
PHCCs primary human cortical cells; abbreviation of the term: primary human cortical cells
HEP heparin
SATB2 a marker protein for mature cortical neurons, abbreviation for the term “Special AT-rich sequence-binding protein 2”
SOX2 transcription factor, abbreviation for “sex-determining region Y box 2”
StarPEG-MMP star-shaped (four-arm) polyethylene glycol, terminally functionalized with enzymatically (matrix metalloprotease) cleavable peptide linkers
Syn synaptophysin
TUBB3 beta-III-tubulin, neuronal cytoplasmic marker

1. A method for forming a functional network of human neuronal and glial cells, comprising the steps of introducing a mixture of cells and polyethylene glycol or heparin are introduced into a synthetic hydrogel system, said hydrogel system containing the components polyethylene glycol (PEG) and heparin and culturing the cells such that during the formation of the three-dimensional hydrogel, the cells are already present in the three-dimensional hydrogel system.

2. The method as claimed in claim 1, wherein the human neuronal cells are cocultured with glial cells and wherein the human neuronal cells are human neuronal stem and progenitor cells or originate from a human immortalized neuronal progenitor cell line or are primary human neuronal progenitor cells obtained from the midbrain.

3. The method as claimed in claim 2, wherein the human neuronal cells are human neuronal stem and progenitor cells from induced pluripotent stem cells (iPSCs) or are derived from primary human cortical cells.

4. The method as claimed in claim 1, wherein functionality of the network of human neuronal and glial cells is determined by expression of mature neuronal cortical markers, by responsiveness to neurotransmitters and by electrophysiological activity.

5. The method as claimed in claim 1, wherein the three-dimensional hydrogel system is a multi-arm polyethylene glycol (star-PEG)-heparin containing hydrogel system which is crosslinked via enzymatically cleavable peptide sequences, wherein the star-PEG-heparin hydrogel system is cleavable and locally reconstructible.

6. The method as claimed in claim 5, wherein the hydrogel matrix of the hydrogel is formed by a covalent crosslinking of a thiol-terminated star-PEG-peptide conjugate and of a heparin functionalized by maleimide, wherein the hydrogel matrix is crosslinked via a Michael addition.

7. The method as claimed in claim 5, wherein the three-dimensional hydrogel matrix of the star-PEG-heparin hydrogel system is formed noncovalently from heparin and a covalent star-PEG-peptide conjugate by self-organization, wherein the star-PEG-peptide conjugate comprises conjugates of two or more peptides which are coupled to a polymer chain and the peptide sequence contains a repeating dipeptide motif (BA)ₙ, where B is an amino acid having a positively charged side chain, A is alanine and n is a number from 5 to 20.

8. The method as claimed in claim 1, wherein the hydrogel has variable mechanical properties, characterized by a storage modulus within a range of 300-600 pascals.

9. The method as claimed in claim 1, wherein the PEG-heparin hydrogel system is modified with signaling molecules and/or with functional peptide units derived from proteins of an extracellular matrix (ECM).

10. The method as claimed in claim 1, that wherein the human neuronal and glial cells are cocultured co-cultured together with human mesenchymal stromal cells and endothelial cells, which are co-localized with the human neuronal and glial cells.

11. A human, neuronal, three-dimensional functional network obtained by the method as claimed in claim 1.

12. A method of monitoring formation of the neuronal network of claim 1 by applying the following steps, quantitatively analyzing the rate of cell growth, as well as length, number and density of branches of the forming network.

13. The method as claimed in claim 12 for monitoring the formation of the neuronal network in real time.

14. The method as claimed in claim 12, further comprising the steps of applying quantitative analysis of connectivity and/or electrophysiological activity of the neuronal cells within the neuronal network.

15. The method as claimed in claim 14 for use of modeling diseases which have an effect on the formation of neurons and/or neuronal networks in the human brain.

16. The method as claimed in claim 15 for the modeling of neurotoxicity and/or change in neuronal stem-cell plasticity caused by disease-relevant protein aggregates.

17. The method as claimed in claim 12 for testing molecules and/or active ingredients which influence neuronal activity and/or network formation.
